(19) United States
(12) Patent Application Publication
(10) Pub. No.: US 2016/0220626 A1
(43) Pub. Date: Aug. 4, 2016

(54) REVERSAL OF INSULIN RESISTANCE AND DYSLIPIDEMIA IN HIGH-FAT DIET-INDUCED OBESE RAT MODELS BY PHYSALIS MINIMA L. EXTRACT

(71) Applicants: Sammer Yousuf, Karachi (PK); Rehana Noshin, Karachi (PK); Mohammad Iqbal Choudhary, Karachi (PK)

(72) Inventors: Sammer Yousuf, Karachi (PK); Rehana Noshin, Karachi (PK); Mohammad Iqbal Choudhary, Karachi (PK)

(21) Appl. No.: 14/609,224

(22) Filed: Jan. 29, 2015

(51) Int. Cl. A61K 36/81

(52) U.S. Cl. CPC A61K 36/81

(57) ABSTRACT

The present invention relates to the effects of Physalis minima L. methanolic extract on dyslipidemia and insulin resistance in insulin resistant high-fat diet-induced obese rats without any systemic toxicities. The extract reversed the dyslipidemia and insulin resistance in high-fat diet-induced obese rats.

### FIELD OF THE INVENTION

The present invention relates to the effects of Physalis minima L. methanolic extract on dyslipidemia and insulin resistance in insulin resistant high-fat diet-induced obese rats without any systemic toxicities. The extract reversed the dyslipidemia and insulin resistance in high-fat diet-induced obese rats.

### BRIEF SUMMARY OF INVENTION

The effect of methanolic extract of Physalis minima L. on body weights, food intake, biochemical parameters, and morphological changes in the liver of high-fat diet-induced insulin resistant obese rat was studied in vivo.

Insulin resistant obese male Wistar rat models were established by feeding high-fat diets for a period of 16 weeks.

Oral application of methanolic extract of Physalis minima caused a significant reduction in the body weight of animals, along with ameliorated dyslipidemia and lowered serum triglyceride and VLDL levels.

The extract further improved the insulin sensitivity, i.e. significantly reduced the fasting blood glucose and serum insulin levels, as well as lowered the hemoglobin A1c levels. Morphological observation by light microscopy displayed a dose-dependent recovery in the hepatic steatosis, caused by prolonged feeding of high-fat diets to the rats.

The application of plant extract caused no differences in the serum urea, creatnine, serum glutamic pyruvic transaminase (SGPT), aspartate aminotransferase (ALT), alkaline phosphatase (ALP), and total and direct bilirubin levels. This indicates that the extract did not exert any adverse effects on renal and liver functions.

The findings clearly demonstrate the anti-obesity potential of methanolic extract of Physalis minima with significant abilities of reversal of dyslipidemia and insulin resistance in vivo (high-fat diet induced obese rat models).

### BRIEF DESCRIPTION OF THE DRAWINGS

FIG. 1A depicts an image of a rat on a normal diet (300 gm body weight).

FIG. 1B depicts the marked difference of weight gain in rats on normal and high-fat diets for 16 weeks.

FIG. 1C depicts the anti-obesity effect of metformin (150 mg/kg body weight for 16 weeks) on high-fat diet group

FIGS. 1D and 1E depict the anti-obesity effect of Physalis minima extracts at concentrations of 150 and 300 mg/kg body weight for 16 weeks, respectively, on a high fat diet

FIG. 2 depicts the effect of a high-fat diet on body weight of rats during 16 weeks of experimental diet.

FIG. 3 depicts serum triglyceride levels after 12 weeks of treatment. The values are expressed as mean±SEM.

FIG. 4 depicts serum VLDL levels after 12 weeks of treatment. The values are expressed as mean±SEM.

FIG. 5 depicts serum total cholestrol levels after 12 weeks of treatment. The values are expressed as mean±SEM.

FIG. 6 depicts fasting blood glucose levels after 12 weeks of treatment. The values are expressed as mean±SEM.

FIG. 7 depicts serum insulin levels after 12 weeks of treatment. The values are expressed as mean±SEM.

FIG. 8A depicts histological examination of the liver with hematoxylin and eosin (H & E) staining; magnification 20× at normal control

FIG. 8B depicts an image of depicts histological examination of the liver with hematoxylin and eosin (H & E) staining; magnification 20× at pathological control.

FIG. 8C depicts an image of liver of animal with metformin (150 mg/kg body weight).

FIGS. 8D and 8E depict an image of Physalis minima at concentrations 150 and 300 mg/kg, respectively.

### DETAILED DESCRIPTION OF INVENTION

Obesity is one of the major risk factors of metabolic disorders (Amin et al., 2012; Rey-López et al., 2014). The prevalence of obesity has dramatically increased across all genders and age groups in developed, as well as in developing countries (Kim et al., 2012). High fat diet is the main source contributing towards obesity (Moreno et al., 2014). Other factors include genetic, environmental, psychological, and physical inactivity (De Angeles et al., 2005). Obesity due to chronic consumption of high-fat diets leads to the development of hypertension, hyperlipidemia, cardiovascular diseases, and insulin resistance (Chothani et al., 2012).

Central obesity (abnormal fat distribution) and insulin resistance are the two major causative features, contributing towards the increasing rate of metabolic syndrome. Other factors, such as hormonal dysregulation, physical inactivity, pro-inflammatory state, ageing, and genetic profile have also been implicated in the development of the metabolic syndrome (Alberti et al., 2005).

A number of rodent models have been developed in order to study the pathogenesis related to the metabolic syndrome. These studies demonstrated that high-fat diet promotes the whole-body insulin resistance and hyperglycemia. The effect of hyperglycemia and insulin resistance on liver physiology, muscle, and insulin signal transduction has also been examined by researchers. These studies indicated that the high-fat diet can be used to cause metabolic syndrome with insulin resistance and compromised the β-cell function in a rodent model (Buettner et al., 2006). High-fat diet, fed to rodents, increases triglyceride levels in muscles followed by insulin resistance, a state equivalent to metabolic syndrome in humans (Sucharitha et al., 2013). Wistar rats fed with high-fat diet are known to develop obesity, hypertension, dyslipidemia, glucose intolerance, and hyperinsulinemia; collectively called metabolic syndrome (Sucharitha et al., 2011).

Most of the pharmacological approaches towards the treatment of obesity are known to possess adverse effects. The discovery of anti-obesity drugs from plants are therefore considered a viable option (Sucharitha et al., 2011).

Physalis minima L. belongs to the family Solanaceae. It is commonly known as ground cherry or sun berry and is found in the tropics of Asia, America, and Africa. Phytochemical analysis showed the major constituents in this plant are sterols, flavonoids, physalins, withanolides, withanones, withaminimins, and alkaloids (Chothani et al., 2012). P. minima is traditionally used as a bitter tonic, diuretic, and laxative. The plant also reported to have anti-inflammatory (Kalsum et al., 2012), analgesic, anti-pyretic, anti-malarial, antibacterial, anti-gonorrheal, anti-lipid peroxidation (DeAngelis et al., 2005), and antileishmanial (Choudhary et al., 2005) activities. Some of the secondary metabolites from this plant also exhibit in vitro amylase, lipase, and alpha-glucosidase inhibitory potentials.

This finding describes the metabolic syndrome reverting and preventing potential of methanol extract of Physalis minima in IFG/IGT obese rats. To the best of our knowledge, this is the first report of in vivo effect of P. minima extract on high fat diet induced obese insulin resistance rat model. Results indicate that P. minima have the potential to reverse dyslipidemia, and insulin resistance in insulin resistant obese rats, without any systemic toxicities.

### Material and Methods

Preparation of extract and metformin. The aerial parts of Physalis minima Linn. were collected from Malir district, Karachi (Pakistan) during August 2012. Plant material was identified by a the taxonomists of the Department of Botany, University of Karachi, and a voucher specimen (G. H. 68261) was submitted in the herbarium of the department. The air dried Physalis minima (20 kg) was crushed and soaked in methanol (20 L). After 5 days, the plant was filtered, followed by evaporation of filtrate under reduced pressure to obtain crude methanolic extract (200 g). The crude gummy material was first defatted by dissolving in petroleum ether, and insoluble part was completely dried and stored at 4° C. The methanolic extract was suspended in distilled water before its administration to the rat model. The doses of the extract were 150 and 300 mg/kg body weight. Metformin was used as a standard drug at a dose of 150 mg/kg body weight for comparison purpose.

Animals. Thirty male Wistar rats (210-220 g) were obtained from the animal house facility of Dr. Panjwani Center for Molecular Medicine and Drug Research (ICCBS). All animals were kept under standard conditions of temperature and humidity with a 12 h light/dark cycle, approved by the Ethical Committee of International Center for Chemical and Biological Sciences (Protocol number: 2013-0001). After one week of acclimation period, rats were randomly divided into two groups. The control group was fed with normal diet (D12450B, Research Diets, USA), whereas the other groups were fed high-fat diet (D12451, Research Diets, USA) for a period of 16-week. The compositions of the diet are given in Table-1 (Boque et al., 2013). After 10 weeks, biochemical parameters were evaluated regularly in order to establish whether the model has developed symptoms of metabolic syndrome.

TABLE 1
Composition of the normal and high-fat diet.

| | |
|---|---|
| D12450B | D12451 |
| (Normal Diet) | (High fat diet) |

| | gm % | Kcal % | gm % | Kcal % |
|---|---|---|---|---|
| Protein | 19.2 | 20 | 24 | 20 |
| Carbohydrate | 67.3 | 70 | 41 | 35 |
| Fat | 4.3 | 10 | 24 | 45 |
| Kcal/gm | 3.85 | | 4.73 | |

Extract supplementation. After a period of 16 weeks, the group of rats on high fat diet (HF) have developed symptoms of metabolic disorders, along with insulin resistance. The HF fed rats were then sub-divided into four groups of three rats each, i.e. Group 1, negative control/pathological control, treated with distilled water; Group 2, positive control, treated with 150 mg/kg of standard drug metformin (Met); Groups 3 and 4, treated groups, received the P. minima extract at doses of 150 and 300 mg/kg, respectively. Normal control rats also received distilled water throughout the treatment for a period of 12 weeks. All the groups, except the control group, were on high-fat diet during the treatment period.

Food intake and body weights. Body weights were recorded weekly throughout the experiment. The food intake was calculated by using following formula, as described by Ghezzi et al, 2012:

$$Food intake = \frac{Daily food intake ( g )}{\sum Body weight of rats in each cage ( g )}$$

Blood and tissue collection and sample preparation. For biochemical analysis after 16 weeks of experimental diet, the blood samples were taken from the tail vein after an overnight fast, whereas after 12 weeks of treatment period, rats were subjected to anesthesia and blood was drawn by cardiac puncture. Liver and pancreas samples were prepared and stained with hematoxylin and eosin, as described by Castro and co-workers. The liver was excised and fixed in buffered formalin. The sectioned liver tissue segments were stained with hematoxylin-eosin (H-E) and observed under light microscope.

Biochemical measurements. Blood glucose, serum insulin, total cholesterol, triglycerides, HDL, LDL, very low density lipoproteins (LDG-LP), urea, creatnine, aspartate aminotransferase (ALT), alkaline phosphatase (ALP), serum glutamic pyruvic transaminase (SGPT), and direct and total bilirubin were estimated. Fasting blood glucose levels were measured using glucometer (Accu Chek Performa, Australia). Fasting insulin levels were quantified using ultra sensitive rat insulin ELISA kit (Crystal Chem, Downers Grove, USA). Insulin sensitivity was evaluated using Homeostatic Model Assessment (HOMA). Serum total cholesterol, triglycerides, HDL, LDL, VLDL, urea, creatnine, AST, ALP, SGPT, and direct and total bilirubins were analysed using automatic analyser (Hitachi, Roche Diagnostics 902, Tokyo, Japan).

Statistical analysis. All data are expressed as mean±SEM. Significance was determined using the two-tailed unpaired student's t test or ANOVA. Differences>less than 0.05 were considered significant.

### Results and Discussion

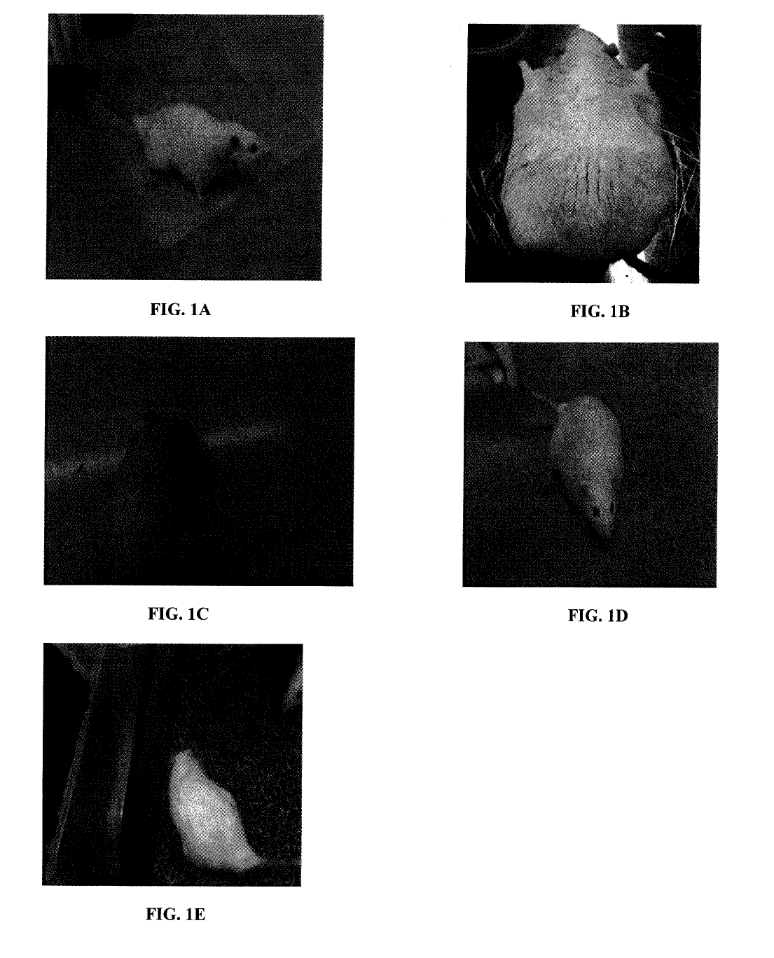

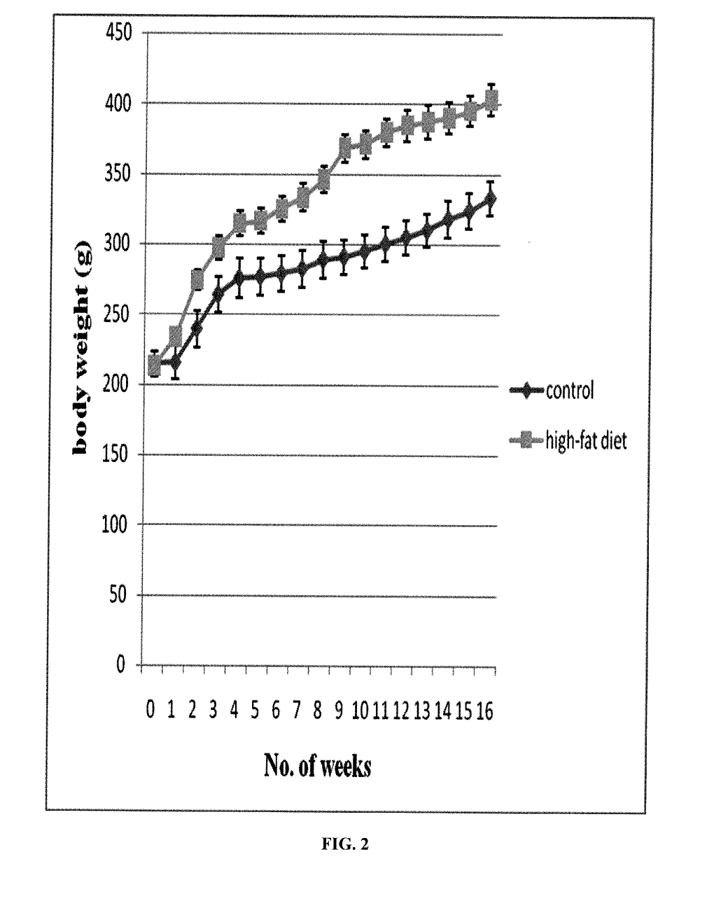

Effect of high-fat diet on physical and biochemical parameters: No difference in the food intake of both groups (control and experimental) was seen throughout the dietary period of 16 weeks. A gradual increase in the body weights of rats fed with high-fat diet was seen (FIGS. 1 and 2). The difference of 32% in body weight of group on high-fat as compared to the control group, clearly indicated the development of obesity, as described by Kelly and co-workers.

The very low density lipoprotein (VLDL), total cholesterol and triglyceride levels in serum were found to be increased to a significant level in group on high-fat diet, as compared to the control group, whereas the low ratio of HDL and LDL in high-fat diet induced obese rat models, as compared to the control rats, further suggested the development of dyslipidemia in high-fat diet fed rats (Table-2).

TABLE 2
Serum lipid profile after 16 weeks of experimental diet.

| | TC | TG | HDL/LDL | VLDL |
|---|---|---|---|---|
| Parameters | (mg/dL) | (mg/dL) | ratio | (mg/dL) |
| Control | 64.09 ± 4.4 | 113.25 ± 7.08 | 4.4 ± 0.5 | 28.18 ± 2.9 |
| High-fat diet | 75.28 ± 3.9 | 227.7 ± 25* | 2.9 ± 0.2 | 46.64 ± 6.04* |

All values are mean ± SE.
*p < 0.05

The fasting blood glucose and serum insulin levels also significantly increased in high-fat diet group, as compared to the control group. The insulin resistance was recorded by observing the levels of HOMA-IR (1.8±0.2 in control group rats, whereas 3.5±0.5 in high-fat diet fed rats). The significant elevated levels of HOMA-IR in obese rat model indicated the development of insulin resistance in high-fat diet-induce obese model. However, no change was observed in HOMA-β level in both groups. ISI represents the insulin sensitivity index which is significantly reduced in the high-fat diet group, suggesting the development of insulin resistance in rats fed with high-fat diet. The results are summarized in Table 3.

TABLE 3
Insulin resistance assessment after 16 weeks of experimental diet.

| | FBG | FIns | | | |
|---|---|---|---|---|---|
| Parameters | (mg/dL) | (ng/mL) | HOMA-IR | HOMA-β | ISI |
| Control | 94.5 ± 1.81 | 0.62 ± 0.06 | 1.8 ± 0.2 | 122.5 ± 3.3 | 56.1 ± 5 |
| High-fat diet | 123.69 ± 2.47* | 1.9 ± 0.2* | 3.5 ± 0.5* | 120.9 ± 6.5 | 28.2 ± 3.1* |

All values are mean ± SE.
*p < 0.05

Effect of extract supplementation on physical and biochemical parameters: Food intake and body weights. Decrease in the average food intake of rats was observed in the metformin and Physalis minima supplemented groups. The group administered daily with the methanolic extract of Physalis minima (150 mg/kg and 300 mg/kg) and standard drug metformin (150 mg/kg) showed a reduction of about 40-70 g and 35 g in body weights, respectively, as compared to the pathological control and control groups, that gained about 109 and 75 g weight, respectively.

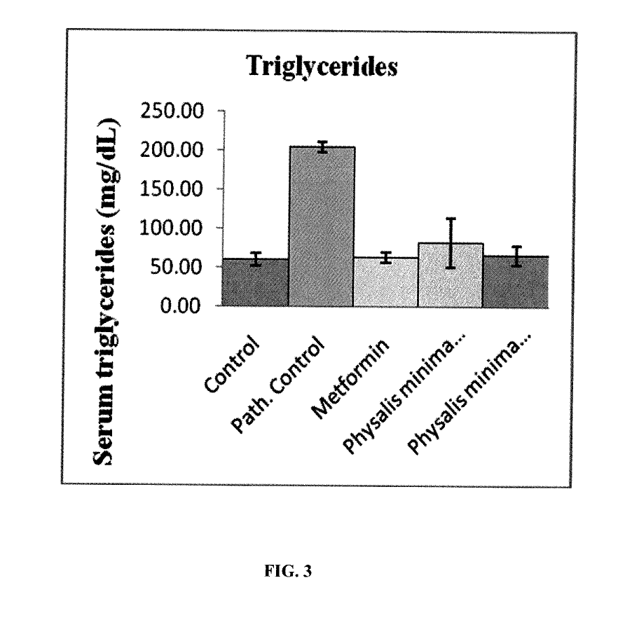

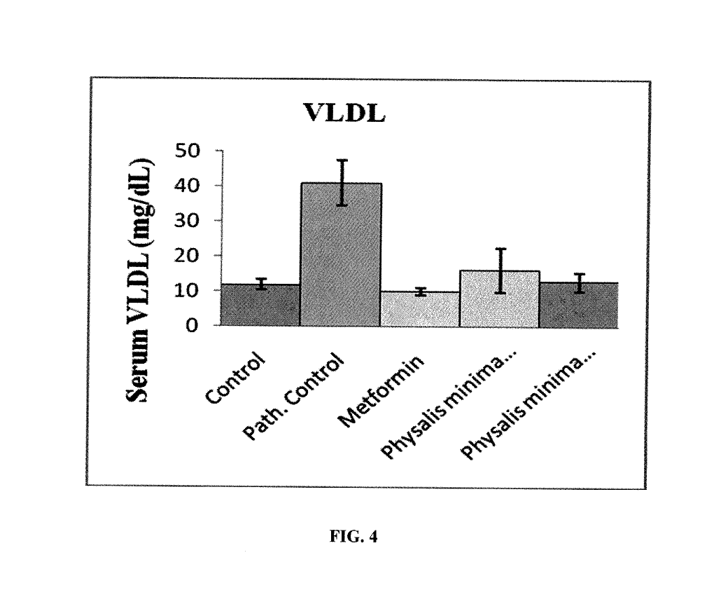

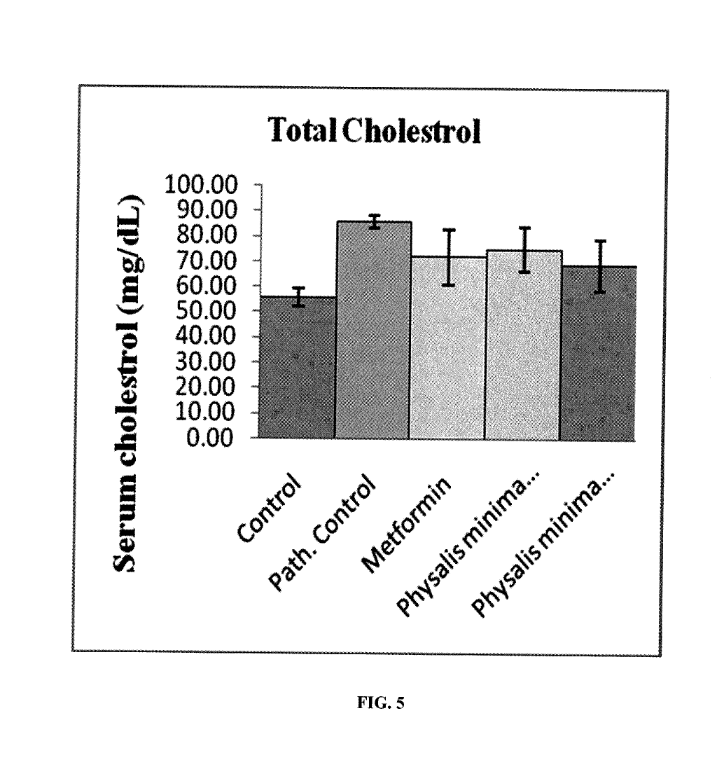

Lipid profile: Significant decrease in the serum triglycerides (FIG. 3) and VLDL levels (FIG. 4) was observed after administration of P. minima extract, whereas no significant differences in the total cholesterol (FIG. 5) and HDL/LDL ratio was seen among the groups. The lipid profile of rats with P. minima extract was improved than the standard drug i.e. metformin group.

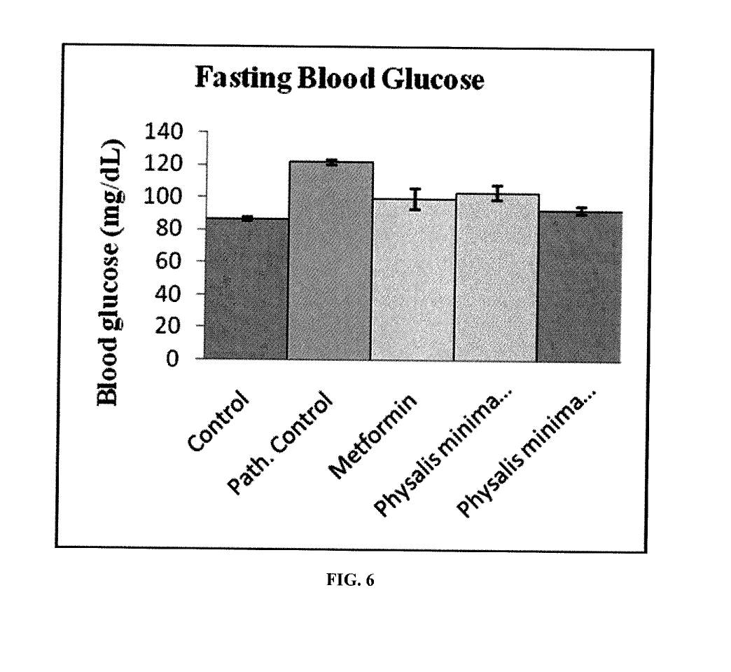

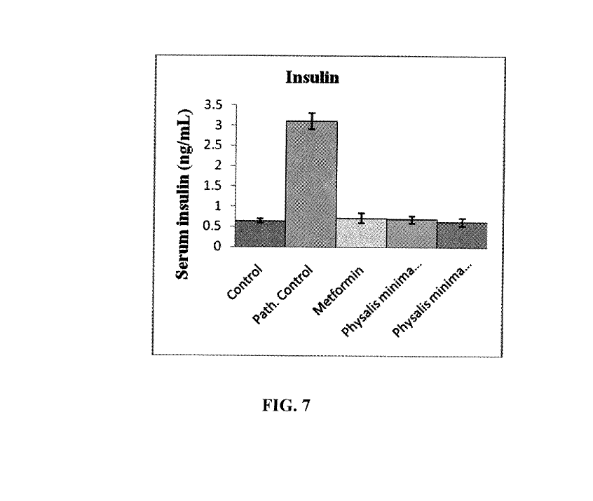

Insulin resistance assessment. P. minima treatment significantly lowered the fasting blood glucose levels, similar to the normal control (FIG. 6). It also improved the insulin sensitivity, observed by decreased serum insulin levels in rats, supplemented with P. minima extract (FIG. 7).

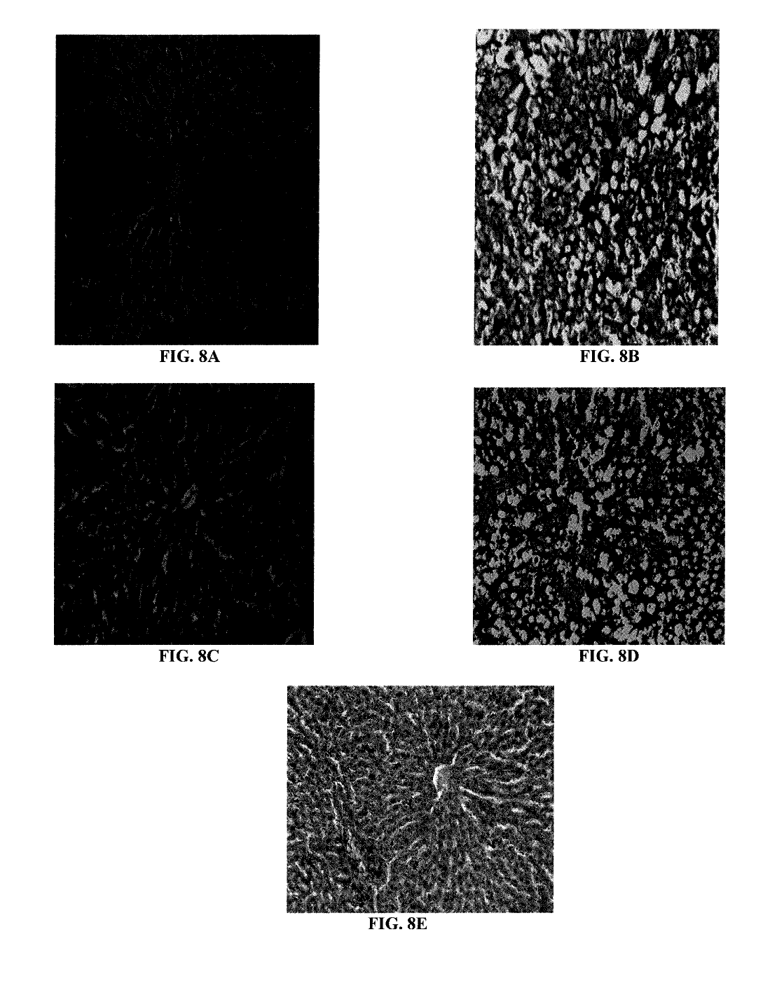

Histopathology of liver. Hepatic morphological changes were examined microscopically by using H&E staining method, as described by Castro UGM et al. It showed an excessive fat accumulation in hepatocytes of pathological control group (FIG. 8B). More severe steatosis was observed in pathological control, as compared to the groups administered with the standard drug metformin (FIG. 8C), and different concentrations of P. minimai.e. 150 mg/kg (FIG. 8D) and 300 mg /kg (FIG. 8E). The development of steatosis, cytoplasmic vacuolation and swelling of hepatocytes exhibited the signs of fatty liver in the pathological group. Supplementation of metformin to the obese rats reversed the condition to the normal. Dose dependent recovery was also seen in the rats, supplemented with P. minima extract.

Evaluation of renal and liver toxicity of P. minima extract: Serum urea, creatnine, aspartate aminotransferase (ALT), alkaline phosphatase (ALP), serum glutamic pyruvic transaminase (SGPT), and total and direct bilirubins were measured to evaluate the toxicity of P. minima towards liver and kidney. The renal (Table 4) and liver functions (Table 5) showed no significant differences after regular treatment with methanolic extract of P. minima, which reflects its safety profile.

TABLE 4
Renal toxicity assessment after 12 weeks of treatment

| | Urea | Creatnine |
|---|---|---|
| Groups | (mg/dL) | (mg/dL) |
| Control | 32.7 ± 0.88 | 0.35 ± 0.02 |
| Pathological control | 27.3 ± 4.6 | 0.43 ± 0.015 |
| Metformin | 31 ± 0 | 0.35 ± 0 |
| Physalis minima (150 mg/kg) | 32 ± 3.05 | 0.36 ± 0.032 |
| Physalis minima (300 mg/kg) | 34 ± 1.15 | 0.37 ± 0.038 |

All values are mean ± SE.
*p < 0.05

TABLE 5
Liver toxicity assessment after 12 weeks of treatment.

| | Total | Direct | | | |
|---|---|---|---|---|---|
| | bilirubin | bilirubin | SGPT | ALP | ALT |
| Groups | (mg/dL) | (mg/dL) | (U/L) | (U/L) | (U/L) |
| Control | 0.29 ± 0 | 0.02 ± 0.005 | 59 ± 6.5 | 44.6 ± 3.38 | 1.67 ± 0.33 |
| Pathological control | 0.29 ± 0.023 | 0.02 ± 0.008 | 32.6 ± 1.45 | 49.66 ± 12.2 | 2 ± 0 |
| Metformin | 0.275 ± 0.025 | 0.02 ± 0 | 48.5 ± 1.5 | 49.5 ± 1.5 | 1.5 ± 0.5 |
| Physalis minima | 0.27 ± 0.02 | 0.02 ± 0.005 | 45.3 ± 4.9 | 64 ± 17.5 | 2 ± 0 |
| (150 mg/kg) | | | | | |
| Physalis minima | 0.27 ± 0.05 | 0.01 ± 0.003 | 39.3 ± 5.81 | 48.3 ± 6.7 | 1.6 ± 0.3 |
| (300 mg/kg) | | | | | |

All values are mean ± SE.
*p < 0.05

What is claimed is:

1. A method for treating insulin resistance in a mammal comprising administering a therapeutically effective amount of an extract of Physalis minima L.

2. The method of claim 1, wherein the mammal has been diagnosed to have metabolic syndrome and the administration of the Physalis minima L. causes a increased insulin sensitivity and lower fasting blood glucose levels.

3. The method of claim 1, wherein the extract is a methanolic extract of Physalis minima.

4. The method according to claim 1, wherein the therapeutically effective amount is administered orally.

5. A method for treating dsyslipidemia in a mammal comprising administering a therapeutically effective amount of an extract of Physalis minima L.

6. The method of claim 5, wherein the mammal has been diagnosed to have metabolic syndrome and the administration of the Physalis minima L. causes a decrease in serum triglyceride, and very low density lipoprotein (VLDL) levels.

7. The method of claim 5, wherein the extract is a methanolic extract of Physalis minima.

8. A method for treating metabolic syndrome in a human comprising administering a therapeutically effective amount of an extract of Physalis minima L. to a human having metabolic syndrome.

9. The method of claim 8, wherein the extract is a methanolic extract.

10. The method of claim 8, wherein the administration of the Physalis minima L. causes increased insulin sensitivity.

11. The method of claim 8, wherein the administration of the Physalis minima L. causes a decrease in serum triglyceride and very low density lipoprotein (VLDL) levels.
